(19) United States
(12) Patent Application Publication
(10) Pub. No.: US 2021/0353489 A1
(43) Pub. Date: Nov. 18, 2021

(54) VIBROTACTILE SUPPORT SYSTEM

(71) Applicant: INNER SPACE INTERFACE, LLC, Phoenix, AZ (US)

(72) Inventors: Paul O'Grady, Pentwater, MI (US); Steven W. Rogers, Portal, AZ (US)

(21) Appl. No.: 15/930,088

(22) Filed: May 12, 2020

(51) Int. Cl. A61H 1/00; A61M 21/00; H04R 1/40; H04R 3/12; H04R 1/02; G10K 11/162; H04R 1/10; A47C 31/02; A47C 7/72; A47C 7/00

(52) U.S. Cl. CPC A61H 1/005; A61H 2201/164; H04R 1/403; H04R 3/12; H04R 1/025; G10K 11/162; H04R 1/10; A47C 31/023; A47C 7/727; A47C 7/004; A61M 2021/0022; A61M 2021/0027; A61H 2201/0149; A61H 2203/0425; A61H 2201/1604; A61M 21/00

(57) ABSTRACT

A vibrotactile support with suppressed distortion and destructive interference having a rigid frame within which is tensioned a high-density material panel in a supportive configuration. A low-durometer, high specific gravity material user interface pad is placed on the high-density panel on the user-supporting side and a plurality of acoustic drivers are attached to the opposing side of the high-density material panel to transmit vibrations through the high-density material panel, the pad, and the user's skin. The acoustic drivers are supplied from an amplifier which is supplied from an electronic audio source. The amplifier may also drive a set of headphones for the user. The audio source may be from any known electronic audio source, including audio-visual presentations. Various shapes and configurations of the vibrotactile support are possible. For non-limiting examples, the vibrotactile support may be configured as a lounge chair, upright chair, baby cradle, or flat bed.

### FIELD OF ART

The present invention relates to a vibrotactile support system for conducting acoustic energy into a supported user's body as well as the user's ears. The present invention more particularly relates to an apparatus for conducting acoustic energy into a supported user's body without distortion or destructive interference.

### BACKGROUND OF THE INVENTION

Systems for conducting sound into a user's body have been produced, but the results are typically less than satisfactory due to distortion and destructive interference of the acoustic waves. The objective of such systems is to provide the user with the same experience of music as the user might experience in a live concert where acoustic waves penetrate the body as well as the ears.

### SUMMARY OF THE INVENTION

The present invention provides a vibrotactile support with suppressed distortion and destructive interference by providing a rigid frame within which is tensioned a high-density panel in a supportive configuration. A high specific gravity and low durometer pad is placed on the high-density panel on the user-supporting side and a plurality of acoustic drivers are attached to the opposing side of the high-density panel. The acoustic drivers are supplied from an amplifier which is supplied from an electronic audio source may also drive a set of headphones for the user. The audio source may be from any known electronic audio source, including audio-visual presentations. Various shapes and configurations of the vibrotactile support are possible. For non-limiting examples, the vibrotactile support may be configured as a lounge chair, upright chair, baby cradle, or flat bed.

### DESCRIPTION OF THE FIGURES OF THE DRAWINGS

The present invention will hereinafter be described in conjunction with the following drawing figures, wherein like numerals denote like elements, and

FIG. 1 is a front-side perspective view illustrating an exemplary embodiment of a vibrotactile support, according to a preferred embodiment of the present invention;

FIG. 2 is an exploded perspective view illustrating the exemplary embodiment of the vibrotactile support of FIG. 1, according to a preferred embodiment of the present invention;

FIG. 3 is a side elevation view illustrating the exemplary embodiment of the vibrotactile support of FIG. 1, according to a preferred embodiment of the present invention;

FIG. 4 is a top plan view illustrating the exemplary embodiment of the vibrotactile support of FIG. 1, according to a preferred embodiment of the present invention;

FIG. 5 is a bottom plan view illustrating the exemplary embodiment of the vibrotactile support of FIG. 1, according to a preferred embodiment of the present invention;

FIG. 6 is a front elevation view illustrating the exemplary embodiment of the vibrotactile support of FIG. 1, according to a preferred embodiment of the present invention;

FIG. 7 is a rear elevation view illustrating the exemplary embodiment of the vibrotactile support of FIG. 1, according to a preferred embodiment of the present invention;

FIG. 8 is a top plan view illustrating an exemplary rim, laid flat, of the exemplary embodiment of the vibrotactile support of FIG. 1, according to a preferred embodiment of the present invention;

FIG. 9 is a top plan view illustrating the exemplary support mat of FIG. 9, laid flat, of the exemplary embodiment of the vibrotactile support of FIG. 1, according to a preferred embodiment of the present invention

FIG. 10 is a top bottom view illustrating the exemplary support mat of FIG. 9, laid flat, showing the locations of acoustic transducers, of the exemplary embodiment of the vibrotactile support of FIG. 1, according to a preferred embodiment of the present invention;

FIG. 11 is a top plan view illustrating the exemplary user interface pad of FIG. 2, laid flat, of the exemplary embodiment of the vibrotactile support of FIG. 1, according to a preferred embodiment of the present invention;

FIG. 12 is a bottom plan view illustrating the exemplary user interface pad of FIG. 2, laid flat, of the exemplary embodiment of the vibrotactile support of FIG. 1, according to a preferred embodiment of the present invention;

FIG. 13 is a diagrammatic longitudinal partial cross-sectional view illustrating the installed order of parts of the exemplary embodiment of the vibrotactile support of FIG. 1, according to a preferred embodiment of the present invention;

FIG. 14 is a top plan view illustrating the exemplary rear acoustic damping cover of FIG. 2, laid flat, of the exemplary embodiment of the vibrotactile support of FIG. 1, according to a preferred embodiment of the present invention;

FIG. 15 is a perspective view illustrating the exemplary rear acoustic damping cover of FIG. 2, in an installed configuration, of the exemplary embodiment of the vibrotactile support of FIG. 1, according to a preferred embodiment of the present invention;

FIG. 16 is a diagrammatic side elevation view illustrating the exemplary support plate of FIG. 1, of the exemplary embodiment of the vibrotactile support of FIG. 1, according to a preferred embodiment of the present invention; and

FIG. 17 is a diagrammatic view illustrating the exemplary embodiment of the vibrotactile support of FIG. 1 in an exemplary vibrotactile system including an audio source, according to a preferred embodiment of the present invention.

### DETAILED DESCRIPTION OF THE INVENTION

In the text below, the hundred's digit(s) of the reference numbers refer to the figure number in regard to which the referenced item is first discussed. Like reference numbers refer to like items. As used and defined herein, the term “vibrotactile” refers to application of vibrations (such as music) to the sense of touch.

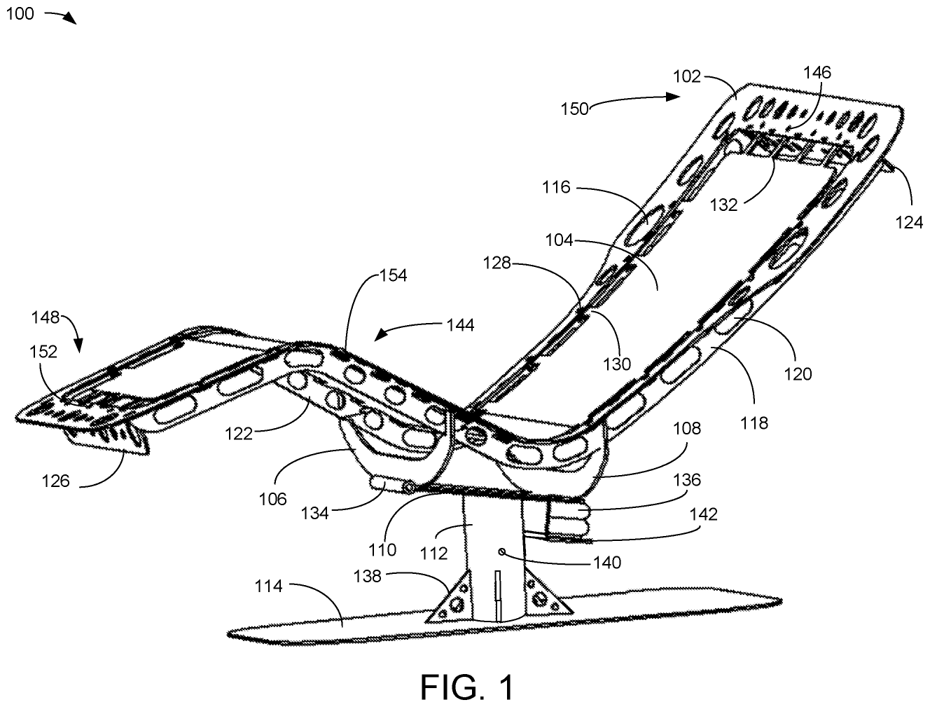

FIG. 1 is a front-side perspective view illustrating an exemplary embodiment of a vibrotactile support 100, according to a preferred embodiment of the present invention. The vibrotactile support 100 is illustrated herein as a lounge chair, but the invention is not so limited. For non-limiting examples, the present invention may be used in theater seating, upright chairs, flat beds, and the like. The wider, upper portion is referred to as the head end 150 and the narrower, lower end is referred to as the foot end 148. Vibrotactile support 100 includes a frame 144, a support mat 104 tensioned within frame 144, a front brace 106, a rear brace 108, a support plate 110, a pedestal 112, and a foot 114.

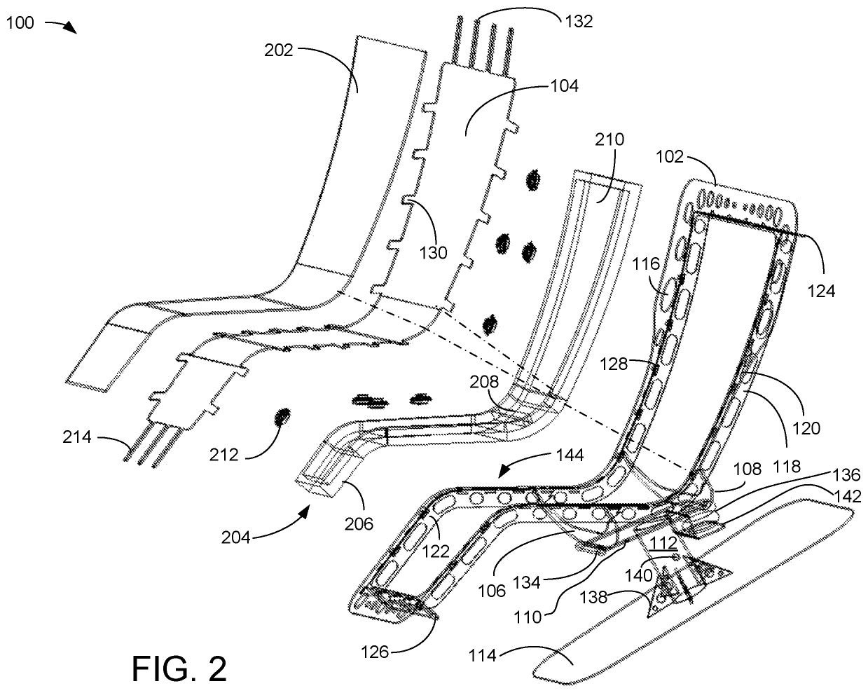

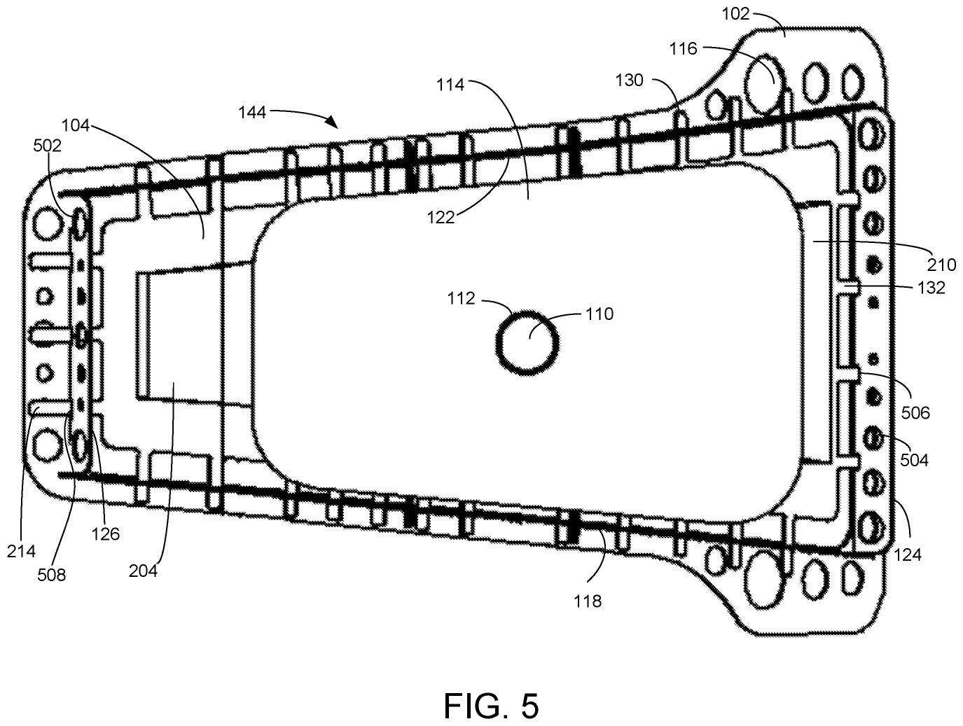

Frame 144 is preferably made of stainless steel. In various embodiments, frame 144 may be made of respectively various materials such as, without limitation, boron-epoxy composite. Frame 144 includes a rim 102 having a plurality of rim openings 116 (one of twenty-five labeled) for adaptive uses and to reduce weight without significant loss of rigidity. Frame 144 also includes left rail 118 and right rail 122 which extend perpendicularly downward from respective left and right sides of rim 102, as shown. Rails 118 and 122 have a plurality of cutout openings 120 for reduction in weight without significant loss of rigidity. Each side tab 130 contains a fastener opening (not shown) and extends through an opening 154 (one of many labeled) in left rail 118 or in right rail 122 to reach the rim side fastener opening 128. Frame 144 also includes foot-end cross brace 126 and head-end cross brace 124 which extend perpendicularly downward from respective foot end 148 and head end 150 of rim 102. Foot-end tabs 214 (see FIG. 2) each contain a fastener opening and they extend, when installed, through tab-admitting openings 508 (see FIG. 5) in foot-end cross brace. Head end tabs 132 each contain a fastener opening and they extend, when installed, through tab-admitting openings 506 (see FIG. 5) in head-end cross brace 124. Rim 102 has rim side fastener opening 128 (one of twenty-four labeled) (twelve on each side) to assist in fastening side tabs 130 (one of twenty-four labeled) (twelve on each side) of support mat 104 to rim 102. Rim 102 has four rim head-end fastener openings 146 (one of four labeled) and three rim foot-end fastener openings 152 (one of three labeled) to assist in fastening head-end tabs 132 (one of four labeled) and foot-end tabs 214 (see FIG. 2, one of three labeled) of support mat 104 to rim 102. In various other embodiments, respective various numbers of tabs 130, 132, and 214 with corresponding rim fastener openings 128, 146, and 152, respectively, may be used.

Front brace 106 is generally U-shaped and rigidly attached to frame 144 on left and right sides of frame 144 and is supported on support plate 110 via hinge 134. Support plate 110 is supported by pedestal 112. The rear end of support plate 110 terminates in a downwardly extending L-shaped support 142, which supports vibration damper 136. Rear brace 108 is generally U-shaped and supported on the vibration damper 136 and is rigidly attached to frame 144 on left and right sides, as shown. Pedestal 112 is illustrated as a right circular cylinder, but that shape is not a limitation of the present invention. Preferably, pedestal 112 is fixed to support plate 110 and to foot 114, as shown. Pedestal 112 has a plurality of support fins 138 (one of three visible of five labeled) fixed to the foot 114. Pedestal 112 also has a set-screw opening 140.

FIG. 2 is an exploded perspective view illustrating the exemplary embodiment of the vibrotactile support 100 of FIG. 1, according to a preferred embodiment of the present invention. Support mat 104 is made of a semi-rigid material. For non-limiting example, a high-density poly Preferably, support mat 104 is made of high-density material. Support mat 104 has twelve side tabs 130 (one of twenty-four labeled) extending from each long side, four long head-end tabs 132 (one of four labeled) extending from the head end 150, and three long foot-end tabs 214 (one of three labeled) extending from the foot end 148. In construction, each side tab on a first long side is aligned over a respective rim side fastener opening 128, the side tab 130 is aligned to the rim side fastener opening 128, and a fastener is inserted through the rim side fastener opening 128 and the side tab 130 to fasten the side tab 130 to the rim 102. Each of the twelve side tabs 130 on the opposing side of the support mat 104 is grasped, in turn, with a locking pliers and pulled toward the opposing side of the rim 102 using a lever to tension the support mat 104. While under tension, each side tab 130 is aligned with a respective rim side fastener opening 128, drilled, and fastened. The procedure is repeated for head-end tabs 132 and foot-end tabs 214, resulting in a tensioned support mat 104 attached within rim 102. Note that the transverse lines shown do not indicate separate pieces, but merely delineate curved sections of support mat 104. Support mat 104 is of one piece.

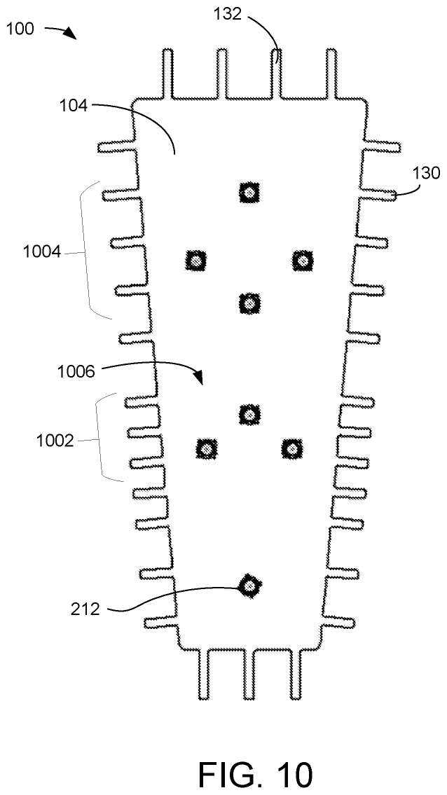

Acoustic transducers 212 are not visible in FIG. 1. Acoustic transducers 212 (one of eight labeled) are adhered to the underside surface 1006 (see FIG. 10) of tensioned support mat 104 and wired for receiving audio signals from an amplifier. In a particular embodiment, the acoustic transducers 212 may be augmented to receive wireless audio signals.

Rear acoustic damping cover 204 is not visible in FIG. 1. Rear acoustic damping cover 204 is adhered to the underside surface 1006 (see FIG. 10) of tensioned support mat 104 over the acoustic transducers 212 to reduce sound transfer to the environment. Adhering is preferably via the use of strips of hook and loop fasteners. Edging 206 and separator bar 208 is made of an acoustical dampening material while back panel 210 is made of a suitable support material. Edging 206 and separator bar 208 are adhered to the back panel 210. Separator bar 208 isolates back noise from the head-end 150 acoustic transducers 212 from foot-end 148 acoustic transducers 212.

User interface pad 202 is preferably a low durometer sheet of high specific gravity material that is preferably held on the top surface 902 (see FIG. 9) of support mat 104 by friction. For non-limiting example, the low durometer sheet of high specific gravity material may be polyethylene. The user interface pad 202 conducts the audio signals transmitted through the support mat 104 from transducers 212 to provide high quality audio signals into the user lying on the user interface pad 202. The user interface pad 202 dampens reflective and destructive interference in the transmitted audio signals, resulting in high-quality vibration transmission of the audio.

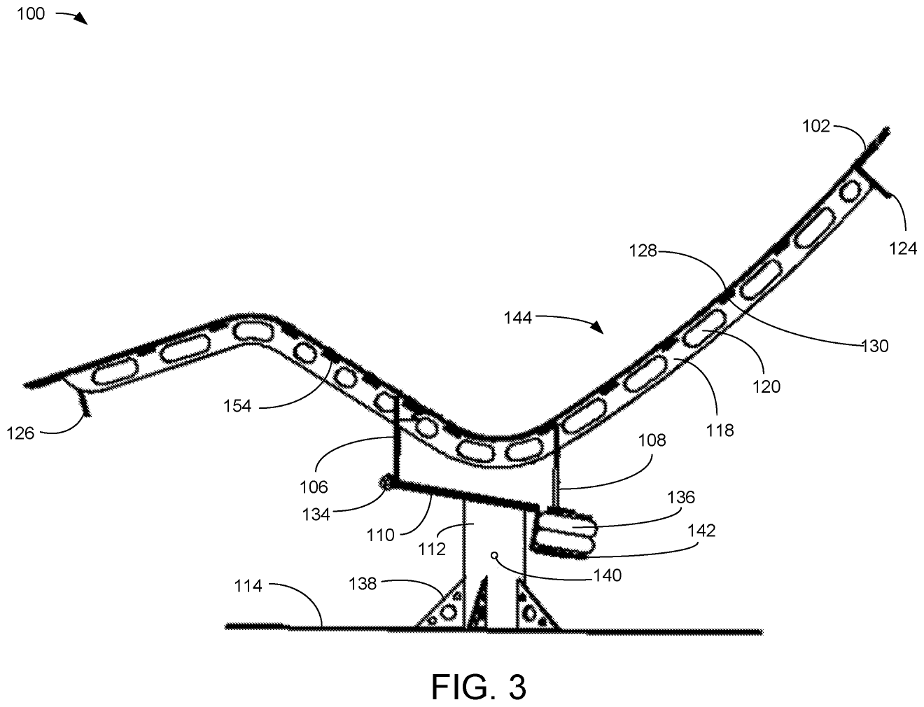

FIG. 3 is a side elevation view illustrating the exemplary embodiment of the vibrotactile support 100 of FIG. 1, according to a preferred embodiment of the present invention. Openings 154 (one labeled of twelve visible of twenty-four) in left rail 118 and right rail 122 allow side tabs 130 to extend through the rails 118 and 122 to reach the rim side fastener openings 128. The illustrated shape of frame 144 is preferred but is not a limitation of the invention. Any shape that can support a user is within the scope of the present invention. In a particular embodiment, the angular orientation of frame 144 relative to pedestal 112 may be adjustable.

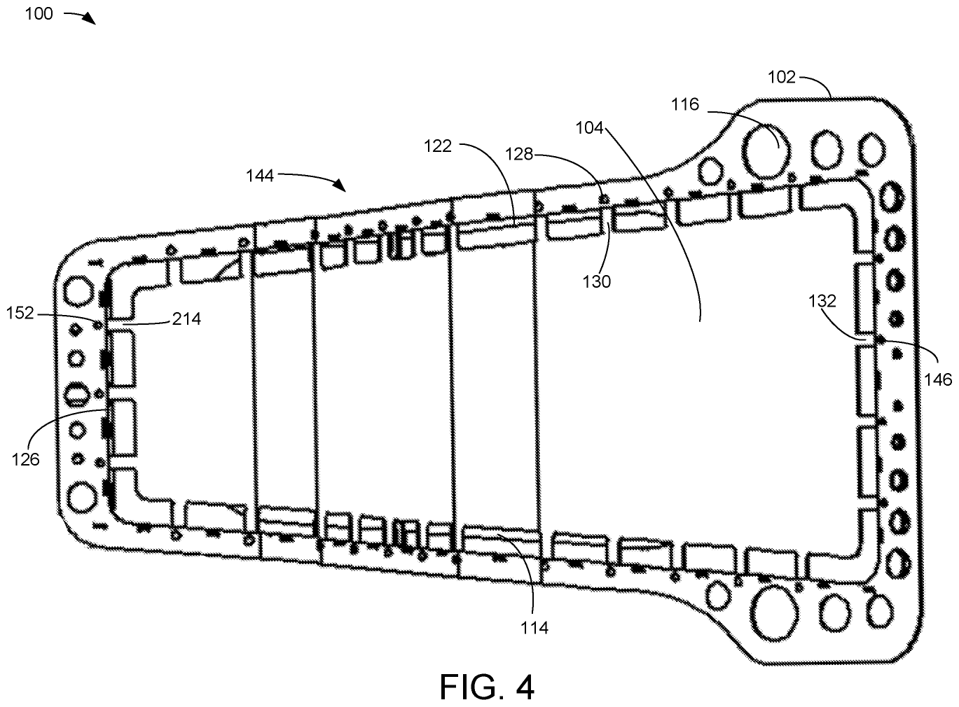

FIG. 4 is a top plan view illustrating the exemplary embodiment of the vibrotactile support 100 of FIG. 1, according to a preferred embodiment of the present invention. The four straight lines transverse to the long axis of the vibrotactile support 100 do not indicate separate pieces; the lines delineate curved portions. The twenty-five rim openings 116 (one of twenty-five labeled) in the rim 102 can best be seen in this view.

FIG. 5 is a bottom plan view illustrating the exemplary embodiment of the vibrotactile support 100 of FIG. 1, according to a preferred embodiment of the present invention. The preferable shape of foot 114 is best seen in this view. In various other embodiments, respectively various shapes may be used. Foot-end cross brace 126 has seven openings 502 (one of seven labeled) for weight reduction without significant loss of rigidity. Head-end cross brace 124 has ten openings 504 (one of ten labeled) for weight reduction without significant loss of rigidity. In various other embodiments, respectively various patterns and/or respective various sizes of frame openings 116, 120, 502, and 504 may be used. Rear acoustic damping cover 204 is shown adhered to support mat 104. Foot-end tabs 214 extend through tab-admitting openings 508 (one of three labeled) through foot-end cross brace 126 to reach rim foot-end fastening openings 152. Head end tabs 132 (one of four labeled) extend through tab-admitting openings 506 (one of four labeled) in head-end cross brace 124 to reach rim head-end fastener openings 146.

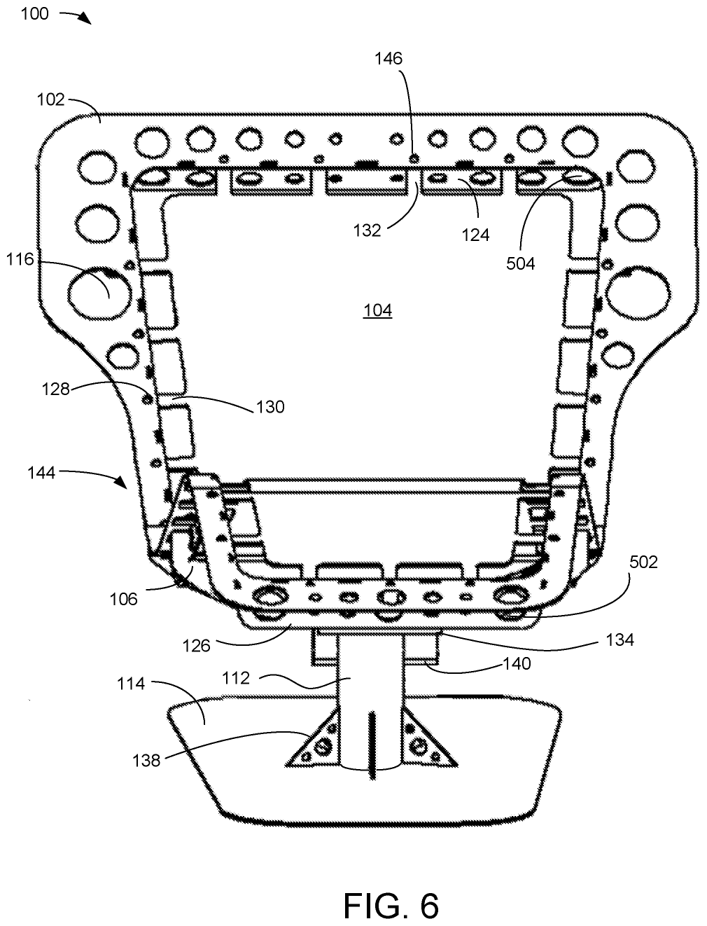

FIG. 6 is a front elevation view illustrating the exemplary embodiment of the vibrotactile support 100 of FIG. 1, according to a preferred embodiment of the present invention. While the illustrated vibrotactile support 100 is for a single user, the invention is not so limited. For example, embodiments sized to hold two or more persons may suffice. In flat bed embodiments, sizes from cradles to king-size beds may be made. In a particular embodiment, the present invention may be integrated into a wheel chair.

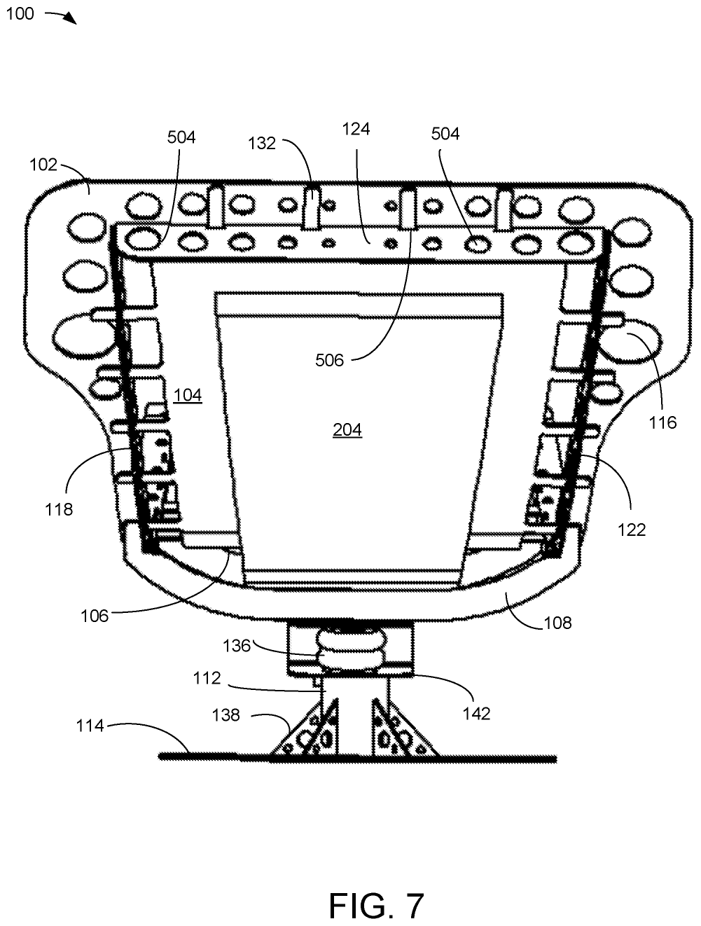

FIG. 7 is a rear elevation view illustrating the exemplary embodiment of the vibrotactile support 100 of FIG. 1, according to a preferred embodiment of the present invention. The position of side tabs 130 and head-end tabs 132 underneath rim 102 can be seen in this view. Fasteners, which are mundane, are not shown.

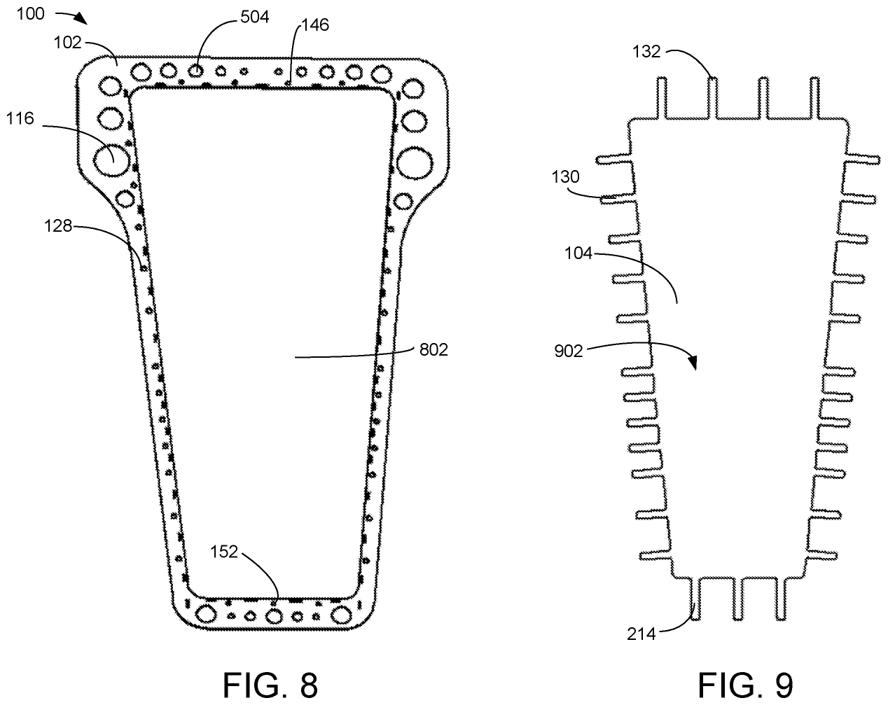

FIG. 8 is a top plan view illustrating an exemplary rim 102, laid flat, of the exemplary embodiment of the vibrotactile support 100 of FIG. 1, according to a preferred embodiment of the present invention. Central opening 802 within rim 102 is sized to accommodate a user on a tensioned support mat 104. All twenty-four rim side fastener openings 128 (one of twenty-four labeled) can be best seen in FIG. 8. All twenty-five rim openings 116 (one of twenty-five labeled) can be best seen in FIG. 8. All four rim head-end fastener openings 146 (one of four labeled) can be best seen in FIG. 8. All three rim foot-end fastener openings 152 (one of three labeled) can be best seen in FIG. 8. While the illustrated shape of rim 102 is preferred, the shape is not a limitation of the present invention. In various embodiments, respectively various shapes and materials may be used, within the constraint of functioning to support a support mat 104 in tension.

FIG. 9 is a top plan view illustrating the exemplary support mat 104 of FIG. 9, laid flat, of the exemplary embodiment of the vibrotactile support 100 of FIG. 1, according to a preferred embodiment of the present invention. Top surface 902 of support mat 104 is preferably free of protrusions and openings. Top surface 902 is preferably frictionally engageable with user interface pad 202. The illustrated shape of support mat 104 is preferred. In various other embodiments, respectively various shapes may be used, including variations in the number and shape of tabs 130, 132, and 214.

FIG. 10 is a bottom plan view illustrating the exemplary support mat 104 of FIG. 9, laid flat, showing the locations of acoustic transducers 212, of the exemplary embodiment of the vibrotactile support 100 of FIG. 1, according to a preferred embodiment of the present invention. Acoustic transducers 212 (one of eight labeled) are grouped on the underside surface 1006 of support mat 104 as a first cluster 1004 of four in the head end 150, a second cluster 1002 for the upper legs of the user, and one acoustic transducer 212 for the lower legs of the user. While this configuration is preferred, various other embodiments may have respectively various configurations of acoustic transducers 212. Acoustic transducers 212 in the preferred embodiment are identical transducers 212, each with a wide frequency range. In various embodiments, respectively various acoustic transducers may be used, such as woofers, tweeters, and combinations of various types of acoustic transducers 212 may be used.

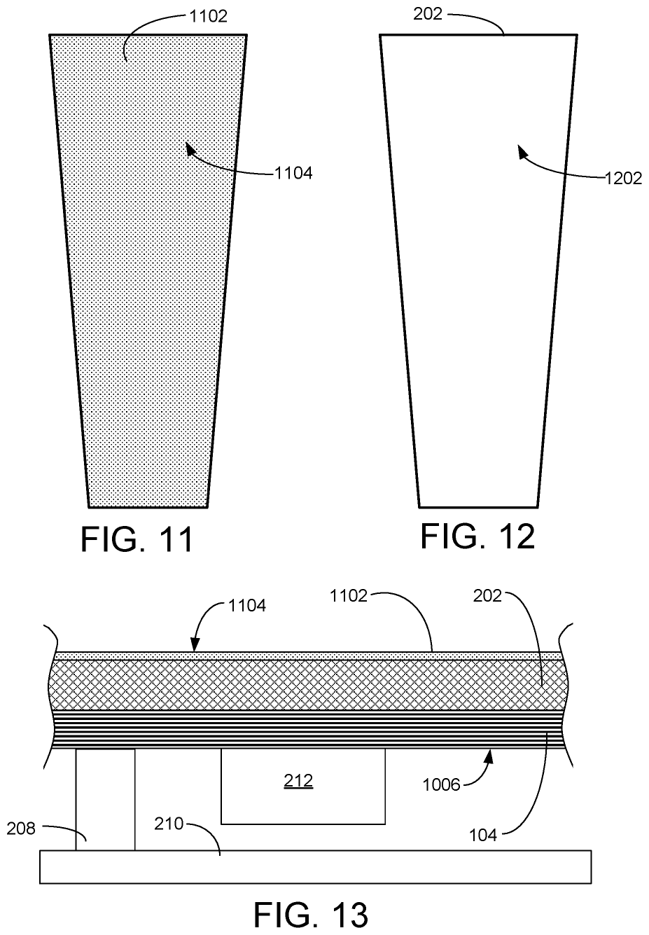

FIG. 11 is a top plan view illustrating the exemplary user interface pad 202 of FIG. 2, laid flat, of the exemplary embodiment of the vibrotactile support 100 of FIG. 1, according to a preferred embodiment of the present invention. Top layer 1102 is preferably fabric, which may be adhered to the user interface pad 202 or the pad may be molded onto the fabric. The user interface pad 202 is primarily a high-density low durometer material. The user interface pad 202 suppresses reflective and destructive interference of the acoustic waves conducted through the support mat 104 from the acoustic transducers 212, and is a novel feature of the present invention. Surface 1104 of the top layer 1102 provides a comparatively low-friction surface 1104 that is suitable for receiving a user.

FIG. 12 is a bottom plan view illustrating the exemplary user interface pad 202 of FIG. 2, laid flat, of the exemplary embodiment of the vibrotactile support 100 of FIG. 1, according to a preferred embodiment of the present invention. Underside surface 1202 of user interface pad 202 is a high-stiction surface 1202. As a result, user interface pad 202 is readily retained on support mat 104 and is releasable from support mat 104 for cleaning and maintenance.

FIG. 13 is a diagrammatic longitudinal partial cross-sectional view illustrating the installed order of parts of the exemplary embodiment of the vibrotactile support 100 of FIG. 1, according to a preferred embodiment of the present invention. FIG. 13 is not to scale. Support mat 104 supports acoustic transducer 212 and separator bar 208 that are releasably attached to underside surface 1006. Back panel 210 of rear acoustic damping cover 204 is fixed to separator bar 208. User interface pad 202 is supported on, and frictionally engaged with, support mat 104. Top layer 1102 provides a low-friction fabric surface 1104 for directly receiving a user.

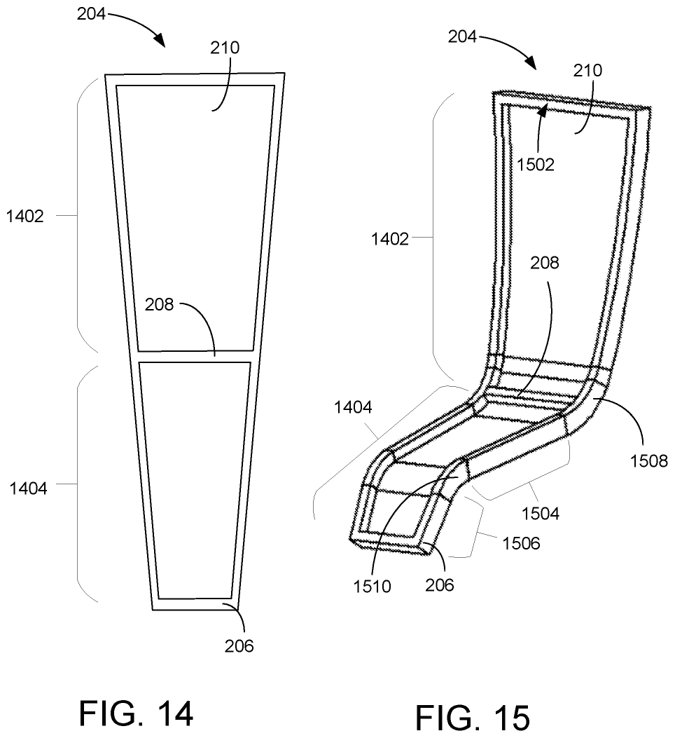

FIG. 14 is a top plan view illustrating the exemplary rear acoustic damping cover 204 of FIG. 2, laid flat, of the exemplary embodiment of the vibrotactile support 100 of FIG. 1, according to a preferred embodiment of the present invention. Separator bar 208 divides the rear acoustic damping cover 204 into an upper portion 1402 and a lower portion 1404 thereby isolating first cluster 1004 of transducers 212 from second cluster 1002 and the lower leg acoustic transducer 212. Rear acoustic damping cover 204 suppresses back pressure sound from acoustic transducers 212 from transmitting into the environment. Separator bar 208 prevents interference between first cluster 1004 acoustic transducers 212 and the remainder of the acoustic transducers 212.

FIG. 15 is a perspective view illustrating the exemplary rear acoustic damping cover 204 of FIG. 2, in an installable configuration, of the exemplary embodiment of the vibrotactile support 100 of FIG. 1, according to a preferred embodiment of the present invention. Separator bar 208 is within the hip curvature 1508, as shown. The upper leg portion 1504 extends from the hip curvature 1508 to the knee curvature section 1510. Lower leg portion 1506 extends from the knee curvature section 1510 to the lower end of rear acoustic damping cover 204. In various other embodiments, respective various curvatures 1508 and 1510 may be used or omitted. Edging 206 has a top surface 1502 that receives an adherent, such as a hook strip or a loop strip of a hook and loop fastener.

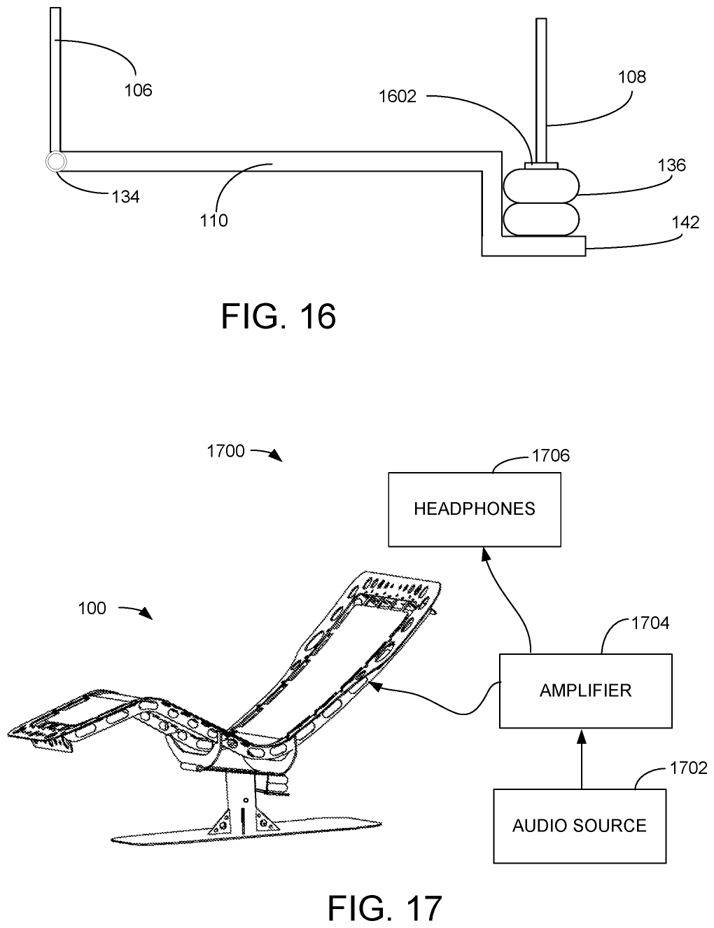

FIG. 16 is a diagrammatic side elevation view illustrating the exemplary support plate 110 of FIG. 1, of the exemplary embodiment of the vibrotactile support 100 of FIG. 1, according to a preferred embodiment of the present invention. Support plate 110 supports hinge 134 to which is attached front brace 106. Support plate 110 terminates in a downwardly extending L-shaped support 142 upon which vibration damper 136 is fixed. Fitting 1602 attaches rear brace 108 to the vibration damper 136. Vibration damper 136 preferably has some intrinsic flexibility, as with a fluid-filled rubber vibration damper 136.

FIG. 17 is a diagrammatic view illustrating the exemplary embodiment of the vibrotactile support 100 of FIG. 1 in an exemplary vibrotactile system 1700 including an audio source 1702, according to a preferred embodiment of the present invention. Audio source 1702 may be any source of recorded or transmitted audio. For non-limiting examples, a television, compact disc player, computer, DVD player, radio, and hard drives are suitable audio sources 1702. Audio is sent from audio source 1702 to amplifier 1704, which may be a plurality of amplifiers and/or multichannel amplifiers. The output of the amplifier 1704 is communicated to the acoustic transducers 212 in the vibrotactile support 100 causing the acoustic transducers 212 to transmit sound vibrations into the user's body. The amplifier 1704 also communicates amplified audio to a set of headphones 1706, to be worn by the user. In a particular embodiment, separate amplifiers may be used for the headphones 1706 and the acoustic transducers 212 in the vibrotactile support 100.

The effect of the present invention is to produce the sensation similar to that of being at a live concert, where the music vibrates the skin as well as the tympanic membrane. While only one embodiment has been illustrated, those of skill in the art, enlightened by the present disclosure, will appreciate the wide variety of possible applications that are within the scope of the claims below.

In the claims below, there may be some functional claiming and there are no statements of intended use.

We claim:

1. A vibrotactile support system comprising:
a. a substantially rigid frame having:
i. a central opening;
ii. a rim; and
iii. a first plurality of rim fastener openings;
b. a support mat having:
i. a perimeter;
ii. a top surface;
iii. a bottom surface; and
iv. a plurality of tabs extending from said perimeter and corresponding to said first plurality of said plurality of rim fastener openings;
c. a second plurality of acoustic transducers releasably attachable to said bottom surface of said support mat; and
d. a low-durometer, high specific gravity material user interface pad releasably engageable with said top surface of said support mat.

2. The vibrotactile support system of claim 1, comprising a left rail and a right rail which extend perpendicularly downward from respective left and right sides of said rim.

3. The vibrotactile support system of claim 2, comprising:
a. a foot end;
b. a foot-end cross brace extending between ends of said left and right rails proximate said foot end;
c. a head end; and
d. a head-end cross brace extending between ends of said left and right rails proximate said head end.

4. The vibrotactile support system of claim 3, comprising:
a. a front brace attached to said frame and flexibly attachable to a support plate; and
b. a rear brace attached to said frame and flexibly attachable to a vibration damper.

5. The vibrotactile support system of claim 4, wherein said vibration damper is mounted on a downwardly extending L-shaped support extending from said support plate.

6. The vibrotactile support system of claim 1, wherein said support mat is operable to be:
a. releasably fastenable within said frame via said plurality of rim fastener openings and said corresponding plurality of tabs; and
b. releasably tensionable within said frame via said plurality of rim fastener openings and said corresponding plurality of tabs.

7. The vibrotactile support system of claim 1, comprising:
a. an acoustic damping cover releasably attachable to said bottom surface of said support mat; and
b. said acoustic damping cover configured to releasably enclose said plurality of acoustic transducers.

8. The vibrotactile support system of claim 7, wherein said acoustic damping cover is segmented via a separator bar.

9. The vibrotactile support system of claim 1, wherein said second plurality of acoustic transducers are arranged in at least two clusters each corresponding to a portion of a user's body.

10. The vibrotactile support system of claim 4, comprising:
a. a plurality of weight-reduction openings in said frame;
b. a plurality of tab-receiving openings in each of said left rail, said right rail, said head-end cross brace, and said foot-end cross brace.

11. The vibrotactile support system of claim 5, comprising:
a. a pedestal supporting said support plate;
b. a foot supporting said pedestal; and
c. a plurality of support fins supported on said foot and on said pedestal.

12. The vibrotactile support system of claim 1, comprising:
a. at least one amplifier in communication with each said acoustic transducer of said plurality of said transducers of said vibrotactile support;
b. an audio source in communication with said at least one amplifier; and
c. a set of headphones in communication with said at least one amplifier.

13. A vibrotactile support system comprising:
a. a substantially rigid frame having:
i. a central opening;
ii. a rim; and
iii. a first plurality of rim side fastener openings;
b. a support mat having:
i. a perimeter;
ii. a top surface;
iii. a bottom surface; and
iv. a plurality of tabs extending from said perimeter and corresponding to said first plurality of said plurality of rim side fastener openings;
c. a second plurality of acoustic transducers releasably attachable to said bottom surface of said support mat;
d. wherein said second plurality of acoustic transducers are arranged in at least two clusters each corresponding to a portion of a user's body;
e. a low-durometer, high specific gravity material user interface pad releasably engageable with said top surface of said support mat;
f. wherein said support mat is operable to be:
i. releasably fastenable within said frame via said plurality of rim fastener openings and said corresponding plurality of tabs; and
ii. releasably tensionable within said frame via said plurality of rim fastener openings and said corresponding plurality of tabs;
g. an acoustic damping cover:
i. releasably attachable to said bottom surface of said support mat; and
ii. configured to releasably enclose said plurality of acoustic transducers.

14. The vibrotactile support system of claim 13, said frame comprising:
a. a left rail and a right rail which extend perpendicularly downward from respective left and right sides of said rim;
b. a foot end operable to support a user's feet;
c. a foot-end cross brace extending between said left and right rails proximate said foot end;
d. a head end operable to support a user's head; and
e. a head end cross brace extending between said left and right rails proximate said head end;
f. a front brace attached to said frame and flexibly attachable to a support plate; and
g. a rear brace attached to said frame and flexibly attachable to a vibration damper.

15. The vibrotactile support system of claim 14, comprising:
a. at least one amplifier in communication with each said acoustic transducer of said plurality of said transducers;
b. an audio source in communication with said at least one amplifier; and
c. a set of headphones in communication with said at least one amplifier.

16. The vibrotactile support system of claim 14, wherein said vibration damper is mounted on a downwardly extending L-shaped support extending from said support plate.

17. The vibrotactile support system of claim 5, comprising:
a. a pedestal supporting said support plate;
b. a foot supporting said pedestal; and
c. a plurality of support fins supported on said foot and on said pedestal.

18. A vibrotactile support system comprising:
a. a substantially rigid frame having:
i. a central opening;
ii. a rim; and
iii. a first plurality of rim fastener openings;
b. a support mat having:
i. a perimeter;
ii. a top surface;
iii. a bottom surface; and
iv. a plurality of tabs extending from said perimeter and corresponding to said first plurality of said plurality of rim side fastener openings;
c. a second plurality of acoustic transducers releasably attachable to said bottom surface of said support mat;
d. wherein said second plurality of acoustic transducers are arranged in at least two clusters each corresponding to a portion of a user's body;
e. a low-durometer, high specific gravity material user interface pad releasably engageable with said top surface of said support mat;
f. wherein said support mat is operable to be:
i. releasably fastenable within said frame via said plurality of rim fastener openings and said corresponding plurality of tabs; and
ii. releasably tensionable within said frame via said plurality of rim fastener openings and said corresponding plurality of tabs;
g. an acoustic damping cover:
i. releasably attachable to said bottom surface of said support mat; and
ii. configured to releasably enclose said plurality of acoustic transducers;

19. The vibrotactile support system of claim 18, said frame comprising:
a. a left rail and a right rail which extend perpendicularly downward from respective left and right sides of said rim.
b. a foot end operable to support a user's feet;
c. a foot-end cross brace extending between said left and right rails proximate said foot end;
d. a head end operable to support a user's head; and
e. a head end cross brace extending between said left and right rails proximate said head end;
f. a front brace attached to said frame and flexibly attachable to a support plate;
g. a rear brace attached to said frame and flexibly attachable to a vibration damper supported on a downwardly extending L-shaped extension of said support plate;
h. a pedestal supporting said support plate;
i. a foot supporting said pedestal; and
j. a plurality of support fins supported on said foot and on said pedestal.

20. The vibrotactile support system of claim 19, comprising:
a. at least one amplifier in communication with each said acoustic transducer of said plurality of said transducers of said vibrotactile support;
b. an audio source in communication with said at least one amplifier; and
c. a set of headphones in communication with said at least one amplifier.
